(19) United States
(12) Patent Application Publication
(10) Pub. No.: US 2024/0268753 A1
(43) Pub. Date: Aug. 15, 2024

(54) Detection of Tissue Damage

(71) Applicant: Bruin Biometrics, LLC, Los Angeles, CA (US)

(72) Inventors: Martin F. BURNS, Los angeles, CA (US); Graham O. ROSS, Oceanside, CA (US)

(73) Assignee: Bruin Biometrics, LLC, Los Angeles, CA (US)

(21) Appl. No.: 18/630,787

(22) Filed: Apr. 9, 2024

Related U.S. Application Data
(63) Continuation of application No. 18/047,084, filed on Oct. 17, 2022, now Pat. No. 11,980,475.
Application No. 18/047,084 is a continuation of application No. 16/271,040, filed on Feb. 8, 2019, now Pat. No. 11,471,094.
(60) Provisional application No. 62/628,676, filed on Feb. 9, 2018.

(51) Int. Cl. A61B 5/00; A61B 5/0531; A61B 5/0537; A61F 5/055; A61F 5/44; A61J 15/00; A61M 5/172; A61M 16/04; A61M 16/06; A61M 25/00

(52) U.S. Cl. CPC A61B 5/447; A61B 5/0531; A61B 5/0537; A61B 5/443; A61B 5/4836; A61B 5/6843; A61B 5/7282; A61F 5/055; A61F 5/44; A61J 15/0003; A61J 15/0084; A61M 5/1723; A61M 16/0465; A61M 16/0497; A61M 16/0666; A61M 25/0017; A61B 2562/0214; A61B 2562/043

(57) ABSTRACT

Methods and apparatus for detection of tissue damage in patients using a medical device for an extended period of time are disclosed.

### CROSS-REFERENCE TO RELATED APPLICATION

This application is a continuation of U.S. Non-Provisional application Ser. No. 18/047,084 filed Oct. 17, 2022, which is a continuation of U.S. Non-Provisional application Ser. No. 16/271,040 filed Feb. 8, 2019, which claims the benefit of U.S. Provisional Application No. 62/628,676, which was filed on Feb. 9, 2018, the entirety of each of which is incorporated herein by reference.

### FIELD

The present disclosure provides methods and apparatus for detecting tissue damage through measurement of Sub-Epidermal Moisture (SEM) and evaluation of those measurements.

### BACKGROUND

The skin is the largest organ in the human body. It is readily exposed to different kinds of damages and injuries. When the skin and its surrounding tissues are unable to redistribute external pressure and mechanical forces, ulcers may be formed. Prolonged continuous exposure to even modest pressure, such as the pressure created by the body weight of a supine patient on their posterior skin surfaces, may lead to a pressure ulcer.

Patients may be required to use a medical device for an extended period of time to treat a particular condition. Some devices are in contact with portions of the patient's body, for example a tube feeding air to a nasal cannula. Patients who are lying prone in a bed may have devices laying on their body, in some cases taped to the skin to hold the device in place. The long-term pressure applied by these devices may be low but the extended period of application may lead to tissue damage that, left untreated, may progress to an open ulcer.

### SUMMARY

In an aspect, the present disclosure provides for, and includes, an apparatus for detecting tissue damage proximate to a point of contact between a medical device and a patient's skin, comprising: a first electrode and a second electrode configured to measure a level of sub-epidermal moisture (SEM) in tissue proximate to the point of contact, an electronics package individually connected to the first and second electrodes and configured to measure a capacitance between the first and second electrodes.

In an aspect, the present disclosure provides for, and includes, a method for detecting tissue damage proximate to a point of contact between a medical device and a patient's skin, comprising the steps of: measuring a plurality of sub-epidermal moisture (SEM) values of tissue proximate to the point of contact at incremental times, comparing the plurality of SEM values, and determining if there is a significant increase in the SEM that indicates that there is tissue damage.

### BRIEF DESCRIPTION OF THE DRAWINGS

Aspects of the disclosure are herein described, by way of example only, with reference to the accompanying drawings. With specific reference now to the drawings in detail, it is stressed that the particulars shown are by way of example and are for purposes of illustrative discussion of aspects of the disclosure. In this regard, the description and the drawings, considered alone and together, make apparent to those skilled in the art how aspects of the disclosure may be practiced.

FIG. 1 depicts a patient wearing a Continuous Positive Airway Pressure (CPAP) mask.

FIG. 2 depicts a patient being treated with a ventilator.

FIG. 3A illustrates the pressure-induced damage associated with a diagnosis of a stage-1 pressure ulcer.

FIG. 3B depicts a patient who has developed a pressure ulcer from a medical device taped to his chest.

FIGS. 3C and 3D depict patients who developed pressure ulcers from urinary catheters.

FIG. 4A depicts a patient wearing a medical device with a Sub-Epidermal Moisture (SEM) sensor, in accordance with the present disclosure.

FIG. 4B depicts a SEM sensing system, in accordance with the present disclosure.

FIG. 5A illustrates how a medical device may contact a patient.

FIG. 5B depicts a SEM sensing device, in accordance with the present disclosure.

FIG. 5C is an enlarged view of a portion of the device of FIG. 5B, in accordance with the present disclosure.

FIG. 6A depicts a patient wearing a medical device that incorporates an elastic retention strap, in accordance with the present disclosure.

FIG. 6B is an enlarged view of a portion of the retention strap of FIG. 6A, in accordance with the present disclosure.

FIGS. 7A and 7B depict example medical devices with controllable pressure management elements, in accordance with the present disclosure.

### DETAILED DESCRIPTION

This description is not intended to be a detailed catalog of all the different ways in which the disclosure may be implemented, or all the features that may be added to the instant disclosure. For example, features illustrated with respect to one embodiment may be incorporated into other embodiments, and features illustrated with respect to a particular embodiment may be deleted from that embodiment. Thus, the disclosure contemplates that in some embodiments of the disclosure, any feature or combination of features set forth herein can be excluded or omitted. In addition, numerous variations and additions to the various embodiments suggested herein will be apparent to those skilled in the art in light of the instant disclosure, which do not depart from the instant disclosure. In other instances, well-known structures, interfaces, and processes have not been shown in detail in order not to unnecessarily obscure the invention. It is intended that no part of this specification be construed to effect a disavowal of any part of the full scope of the invention. Hence, the following descriptions are intended to illustrate some particular embodiments of the disclosure, and not to exhaustively specify all permutations, combinations, and variations thereof.

Unless otherwise defined, all technical and scientific terms used herein have the same meaning as commonly understood by one of ordinary skill in the art to which this disclosure belongs. The terminology used in the description of the disclosure herein is for the purpose of describing particular aspects or embodiments only and is not intended to be limiting of the disclosure.

All publications, patent applications, patents and other references cited herein are incorporated by reference in their entireties for the teachings relevant to the sentence and/or paragraph in which the reference is presented. References to techniques employed herein are intended to refer to the techniques as commonly understood in the art, including variations on those techniques or substitutions of equivalent techniques that would be apparent to one of skill in the art.

U.S. patent application Ser. No. 14/827,375 discloses an apparatus that measures the sub-epidermal capacitance using a bipolar sensor, where the sub-epidermal capacitance corresponds to the moisture content of the target region of skin of a patient. The '375 application also discloses an array of these bipolar sensors of various sizes.

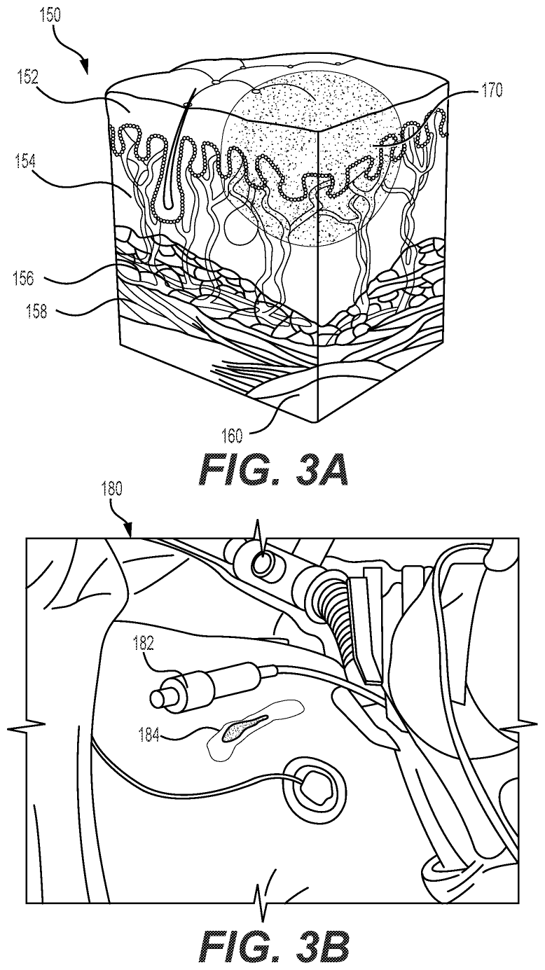

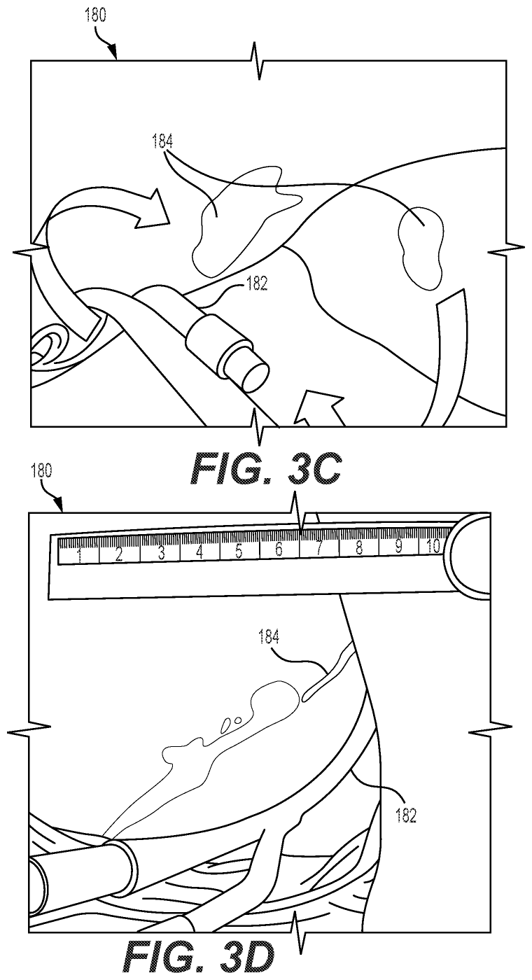

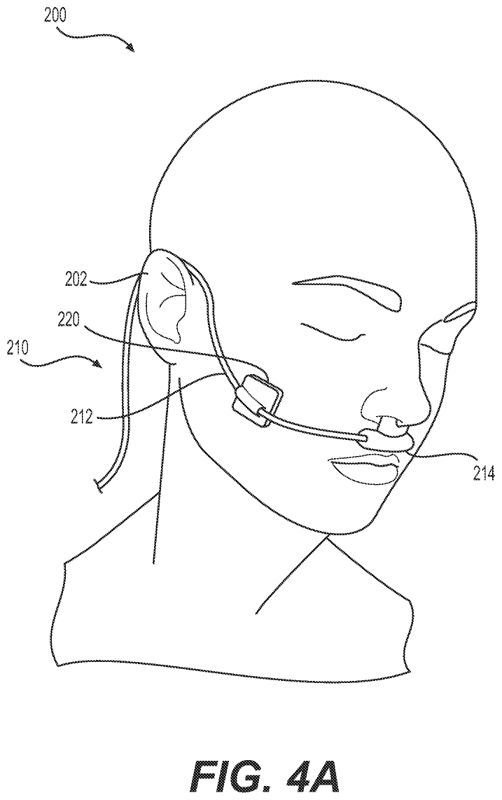

U.S. patent application Ser. No. 15/134,110 discloses an apparatus for measuring sub-epidermal moisture (SEM) similar to the device shown in FIG. 3, where the device emits and receives an RF signal at a frequency of 32 kHz through a single coaxial sensor and generates a bioimpedance signal, then converts this signal to a SEM value.

Both U.S. patent application Ser. Nos. 14/827,375 and 15/134,110 are incorporated herein by reference in their entireties.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted.

The methods disclosed herein include and comprise one or more steps or actions for achieving the described method. The method steps and/or actions may be interchanged with one another without departing from the scope of the present disclosure. In other words, unless a specific order of steps or actions is required for proper operation of the embodiment, the order and/or use of specific steps and/or actions may be modified without departing from the scope of the present disclosure.

As used in the description of the disclosure and the appended claims, the singular forms “a,” “an,” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a length, a frequency, or a SEM value and the like, is meant to encompass variations of ±20%, ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

As used herein, the term “sub-epidermal moisture” or “SEM” refers to the increase in tissue fluid and local edema caused by vascular leakiness and other changes that modify the underlying structure of the damaged tissue in the presence of continued pressure on tissue, apoptosis, necrosis, and the inflammatory process.

As used herein, a “patient” may be a human or animal subject.

As used herein, “delta” refers to a calculated difference between two SEM values.

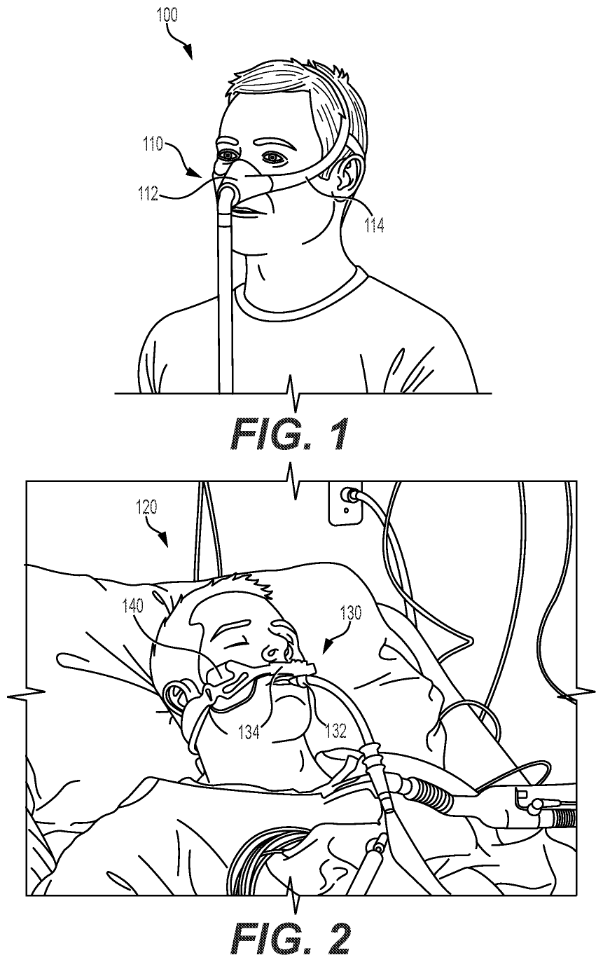

FIG. 1 depicts a patient 100 wearing a CPAP mask 110. A CPAP system is used by individuals having difficulty in breathing while sleeping, among others. The mask 110 is worn every night for the entire time that the person is asleep, typically 7-9 hours. This repeated exposure of sensitive facial tissue, where the skin is close to bone, to long-duration low-pressure contact by the nosepiece 112 or straps 114 poses a risk of developing a pressure ulcer.

FIG. 2 depicts a patient 120 being treated with a ventilator, which includes mouthpiece 130 having, in this example, an endotracheal tube 132 held in place by a strap 134. Patients that are unable to breathe satisfactorily on their own are “put on” a respirator to ensure that their body is receiving sufficient oxygen to heal. A patient may be on a ventilator for a few hours or a few weeks, depending on the injury. Patients who are on a ventilator for extended periods of time may be put in a medically induced coma because of the discomfort of the ventilator, further reducing their mobility and increasing the risk of a pressure ulcer. In FIG. 2, a pad 140 has been placed on the cheek of the patient 120 and under the strap 134 in order to distribute pressure and protect the skin.

FIG. 3A illustrates the pressure-induced damage associated with a diagnosis of a stage-1 pressure ulcer. This cutaway view of a section of skin tissue 150 shows the top layer stratum corneum, the dermis 154, a layer of fat 156 over a layer of muscle 158, and a bone 160. The darkened region 170 indicates damage to the skin penetrating from the stratum corneum 152 down into the dermis 154. The surface of the skin over region 170 may show a redness and a difference in firmness that can be identified by a trained clinician as a symptom of the damage.

FIG. 3B depicts a patient 180 who has developed a pressure ulcer 184 from a medical device 182 taped to his chest. FIG. 3C depicts a patient 180 who has developed a pressure ulcer 184 in the pubic area from a medical device 182, which is a urinary tube. FIG. 3D depicts a patient 180 who has developed a pressure ulcer 184 in the lower abdomen area from a medical device 182, which is also a urinary tube. Development of this type of injury depends on many factors, including the amount of local pressure on the skin, whether additional pressure was created by other items laying over the device 182, and the duration of the pressure. Development of an ulcer is also affected by the condition of the patient's skin, which depends on the age of the patient and their health.

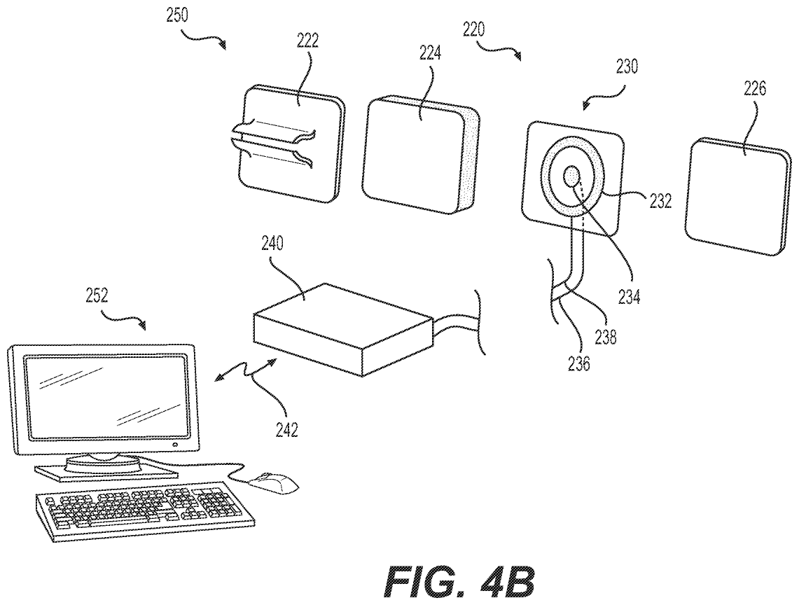

FIG. 4A depicts a patient 200 wearing a medical device 210 with a Sub-Epidermal Moisture (SEM) sensor (not visible in FIG. 4A), in accordance with the present disclosure. There is contact between the device 210 and the patient 200 in multiple locations, such as behind the ear, along the tube 212 over the cheek, at the location of retention device 220, and at the fitting 214 where the tube 212 connects to a nasal cannula (not visible in FIG. 4A). In general, tension on the tube 212 creates pressure in many if not all of these locations.

FIG. 4B depicts an example SEM sensing system 250, in accordance with the present disclosure. The system 250 includes a molded plastic clip 222 configured to attach to the tube 212, a layer of foam 224 to distribute pressure, a SEM sensor 230. In an aspect, there is a layer of adhesive 226 to attach the retention device 220 to the skin of the patient 200. The sensor 230 has electrodes 232, 234 that are connected via wires 236, 238 to electronics package 240, which is configured to make a measurement of the capacitance between the two electrodes 232, 234 and calculate a “delta” value that is, in one aspect, the difference between the highest SEM value and the lowest SEM value in a set of measurements. In an aspect, a set of measurements is taken during a single clinical evaluation. In one aspect, a set of measurements is taken over time, with the first measurement taken at the time of the first use of the medical device.

In an aspect, a calculated delta value is compared to a threshold. When the delta value exceeds the threshold, this indicates a degree of damage. There may be multiple thresholds used to evaluate multiple levels of tissue damage. In one aspect, the maximum SEM value is compared to a threshold. When the maximum value exceeds the threshold, this indicates a degree of damage.

In an aspect, a threshold may be about 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5. In one aspect, a threshold may range from 0.1 to 8.0, such as from 0.1 to 1.0, from 1.1 to 2.0, from 2.1 to 3.0, from 3.1 to 4.0, from 4.1 to 5.0, from 5.1 to 6.0, from 6.1 to 7.0, from 7.1 to 8.0, from 0.1 to 7.5, from 0.5 to 8.0, from 1.0 to 7.0, from 1.5 to 6.5, from 2.0 to 6.0, from 3.0 to 5.5, from 3.5 to 5.0, or from 4.0 to 4.5. In an aspect, a threshold can be scaled by a factor or a multiple based on the values provided herein. It will be understood that a threshold is not limited by design, but rather, one of ordinary skill in the art would be capable of choosing a predetermined value based on a given unit of SEM. In one aspect, thresholds of the present disclosure are varied according to the specific portion of a patient's body on which measurements are being made, or one or more characteristics of the patient such as age, height, weight, family history, ethnic group, and other physical characteristics or medical conditions.

In an aspect, the electronics package 240 includes devices to communicate over link 242 to computer 252, which may be a PC, a mobile tablet, a mobile phone, a server using cloud-based data storage and analysis, or other data systems. Link 242 may include a wired or wireless communication element, optical communication elements, a network that may have one or more switches and routers, and other standard data transfer devices and protocols. Link 242 may also be implemented as hardware with nonvolatile storage, for example a “thumb drive,” that is loaded with data by the electronics package 240 and in turn is physically relocated and connected to the computer 252 whereupon it delivers the data. In an aspect, Link 242 provides real-time communication of recorded SEM measurements and/or calculated delta values from electronic package 240 to computer 252 to allow for real-time monitoring of ulcer development in a patient.

In one aspect, a molded plastic clip 222 of SEM sensing system 250 of the present disclosure is configured to attach to a medical device selected from the group consisting of a nasogastric tube, a feeding tube, an endotracheal tube, a tracheostomy tube, a tracheostomy collar, a nasal cannula, an IV/PICC line, a central line, a catheter, and a fecal management tube. In an aspect, adhesive 226 has a shape selected from the group consisting of substantially a square, substantially a rectangle, substantially a circle, and a polygon. In one aspect, a face of adhesive 226 has a surface area less than 25 cm², such as less than 20 cm², less than 15 cm², less than 10 cm², or less than 5 cm². In an aspect, SEM sensing system 250 has a mass of less than 5 grams, such as less than 4 grams, less than 3 grams, less than 2 grams, less than 1 gram, or less than 0.5 gram.

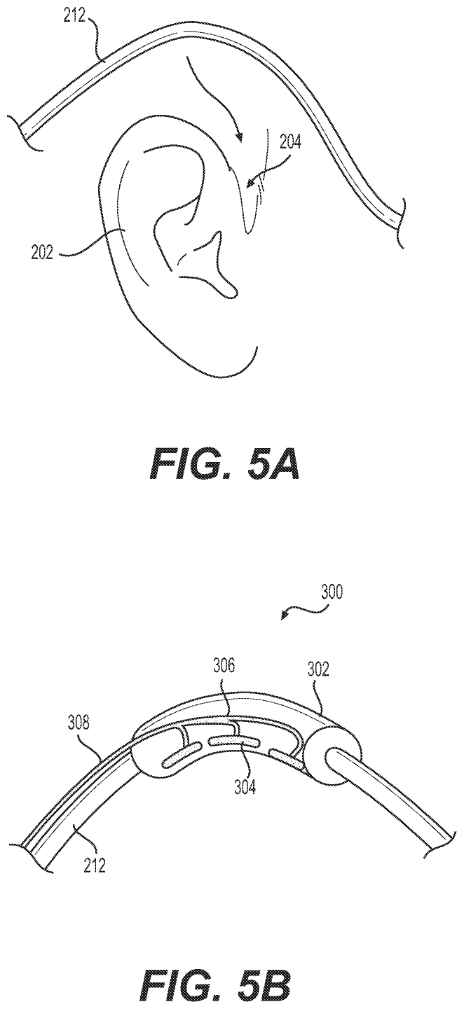

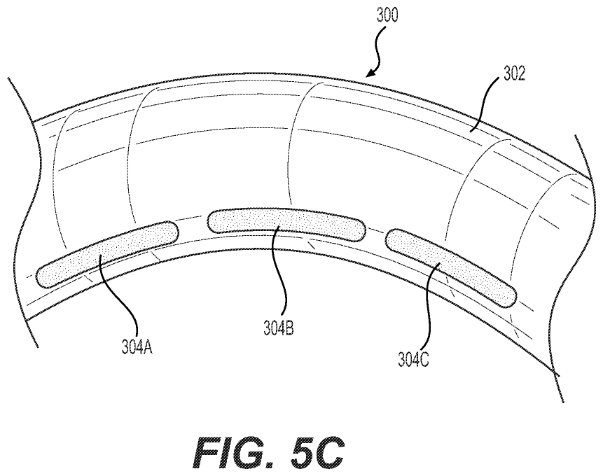

FIG. 5A illustrates how a medical device may contact a patient. The tube 212 from FIG. 4A runs over the crease 204 between a patient's ear 202 and their skull. Pressure can develop at the point of contact between tube 212 and the crease 204 due to tension in tube 212.

FIG. 5B depicts a SEM sensing device 300, in accordance with the present disclosure. In an aspect, the device 300 is added to a basic medical device, for example tube 212. Electrodes 304 on the external surface of the device body 302 are connected by wires 306 of cable 308 to an external electronics package (not shown in FIG. 5B). In an aspect, the device 300 comprises a processor (not visible in FIG. 5B) that does one or more of switching, sensing, and measurement. In an aspect, the processor provides wireless communication to the electronics package. In one aspect, the wireless communication to the electronics package from the electrodes occurs in real-time. In an aspect, the wireless communication to the electronics package is delayed.

FIG. 5C is an enlarged view of a portion of the device 300 of FIG. 5B, in accordance with the present disclosure. In this example, there are three electrodes 304A, 304B, and 304C that are aligned in a row on the surface of body 302, but this array of electrodes may utilize two or more electrodes that are disposed in any two-dimensional pattern. In an aspect, device 300 may comprise three or more electrodes, such as four or more electrodes, five or more electrodes, ten or more electrodes, fifteen or more electrodes, twenty or more electrodes, twenty-five or more electrodes, thirty or more electrodes, forty or more electrodes, or fifty or more electrodes.

In FIG. 5C, electrodes 304A, 304B, 304C are elongated rectangles with rounded ends, but these electrodes may be provided in any shape and size. In an aspect, electrodes 304A, 304B, and 304C may be any shape or configuration, such as point electrodes, plate electrodes, ring electrodes, hexagonal electrodes, or interdigitated finger electrodes. In this example, the long, thin aspect ratio of the electrodes over the curved body 302 provides for complete contact between each electrode 304A, 304B, 304C and the patient's skin. In one aspect, electrodes of device 300 are approximately evenly spaced apart by from about 0.1 cm to about 5 cm, such as from about 0.2 cm to about 5 cm, from about 0.3 cm to about 5 cm, from about 0.4 cm to about 5 cm, from about 0.5 cm to about 5 cm, from about 1 cm to about 5 cm, from about 1.5 cm to about 5 cm, from about 2 cm to about 5 cm, from about 2.5 cm to about 5 cm, from about 3 cm to about 5 cm, from about 3.5 cm to about 5 cm, from about 4 cm to about 5 cm, from about 4.5 cm to about 5 cm, from about 0.1 cm to about 4.5 cm, from about 0.1 cm to about 4 cm, from about 0.1 cm to about 3.5 cm, from about 0.1 cm to about 3 cm, from about 0.1 cm to about 2.5 cm, from about 0.1 cm to about 2 cm, from about 0.1 cm to about 1.5 cm, from about 0.1 cm to about 1 cm, from about 0.1 cm to about 0.9 cm, from about 0.1 cm to about 0.8 cm, from about 0.1 cm to about 0.7 cm, from about 0.1 cm to about 0.6 cm, from about 0.1 cm to about 0.5 cm, from about 0.1 cm to about 0.4 cm, from about 0.1 cm to about 0.3 cm, from about 0.1 cm to about 0.2 cm, from about 0.5 cm to about 4.5 cm, from about 1 cm to about 4 cm, from about 1.5 cm to about 3.5 cm, or from about 2 cm to about 3 cm. In an aspect, there is an insulating cover layer over each of the electrodes 304A, 304B, 304C.

Still referring to FIG. 5C, the electrodes 304A, 304B, 304C are individually coupled to the electronics package or other controlling processor such that pairs of any two electrodes may be selected to form a two-electrode sensor. With an array of electrodes, a plurality of sensors may be formed to measure capacitance over a region without moving the device 300. For example, electrodes 304A, 304B can be paired to measure the SEM in the tissue between the electrodes 304A, 304B, then electrodes 304B, 304C can be paired to measure the SEM in the tissue between the electrodes 304B, 304C.

In an aspect, device 300 of the present disclosure is configured to attach to a medical device selected from the group consisting of a nasogastric tube, a feeding tube, an endotracheal tube, a tracheostomy tube, a nasal cannula, an IV/PICC line, a central line, a catheter, and a fecal management tube. In one aspect, device 300 has a mass of less than 5 grams, such as less than 4 grams, less than 3 grams, less than 2 grams, less than 1 gram, or less than 0.5 gram.

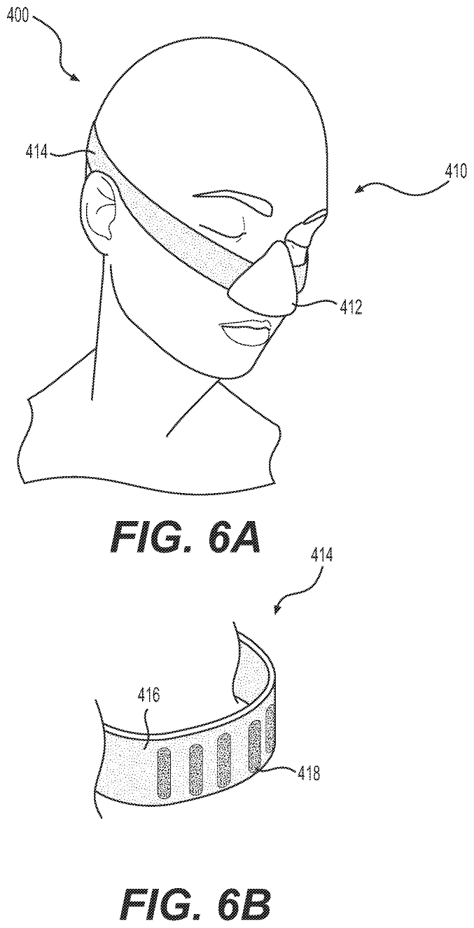

FIG. 6A depicts a patient 400 wearing a medical device 410 that incorporates a retention strap 414 to hold nosepiece 412 in place, in accordance with the present disclosure. In order to function, there must be tension in the elastic strap 414 and along the contact edges of nosepiece 412.

FIG. 6B is an enlarged view of a portion of the retention strap 414 of FIG. 6A, in accordance with the present disclosure. In this example, electrodes 418 are attached to the elastic 416 such that the electrodes 418 are in contact with the patient's skin while the device 410 is worn. In one aspect, electrodes 418 are elongated-shaped electrodes. In an aspect, similar electrodes (not shown in FIG. 6B) are located on the contact surface of the nosepiece. As described with respect to FIG. 5C, the individual electrodes of an array of electrodes 418 can be connected in various pairs to form sensors. In an aspect, the retention strap 414 includes one or more of a battery, a processor, data storage, and a communication element.

In an aspect, retention strap 414 may comprise two or more electrodes, such as three or more electrodes, four or more electrodes, five or more electrodes, ten or more electrodes, fifteen or more electrodes, twenty or more electrodes, twenty-five or more electrodes, thirty or more electrodes, forty or more electrodes, fifty or more electrodes or a hundred or more electrodes.

In one aspect, electrodes of retention strap 414 are approximately evenly spaced apart by from about 0.1 cm to about 5 cm when the retention strap is in a relaxed state, such as from about 0.2 cm to about 5 cm, from about 0.3 cm to about 5 cm, from about 0.4 cm to about 5 cm, from about 0.5 cm to about 5 cm, from about 1 cm to about 5 cm, from about 1.5 cm to about 5 cm, from about 2 cm to about 5 cm, from about 2.5 cm to about 5 cm, from about 3 cm to about 5 cm, from about 3.5 cm to about 5 cm, from about 4 cm to about 5 cm, from about 4.5 cm to about 5 cm, from about 0.1 cm to about 4.5 cm, from about 0.1 cm to about 4 cm, from about 0.1 cm to about 3.5 cm, from about 0.1 cm to about 3 cm, from about 0.1 cm to about 2.5 cm, from about 0.1 cm to about 2 cm, from about 0.1 cm to about 1.5 cm, from about 0.1 cm to about 1 cm, from about 0.1 cm to about 0.9 cm, from about 0.1 cm to about 0.8 cm, from about 0.1 cm to about 0.7 cm, from about 0.1 cm to about 0.6 cm, from about 0.1 cm to about 0.5 cm, from about 0.1 cm to about 0.4 cm, from about 0.1 cm to about 0.3 cm, from about 0.1 cm to about 0.2 cm, from about 0.5 cm to about 4.5 cm, from about 1 cm to about 4 cm, from about 1.5 cm to about 3.5 cm, or from about 2 cm to about 3 cm.

In an aspect, retention strap 414 of the present disclosure is configured to function as a tracheostomy strap. In one aspect, retention strap 414 of the present disclosure is configured to function as an abdominal binder. In an aspect, retention strap 414 of the present disclosure is configured to attach to an oxygen delivery mask. In one aspect, retention strap 414 of the present disclosure is configured to attach to an identification band.

In one aspect, a face of retention strap 414 has a surface area less than 6000 cm², such as less than 5000 cm², less than 4000 cm², less than 3000 cm², less than 2000 cm², less than 1000 cm², less than 500 cm², less than 100 cm², less than 50 cm², less than 25 cm², less than 20 cm², less than 15 cm², less than 10 cm², or less than 5 cm².

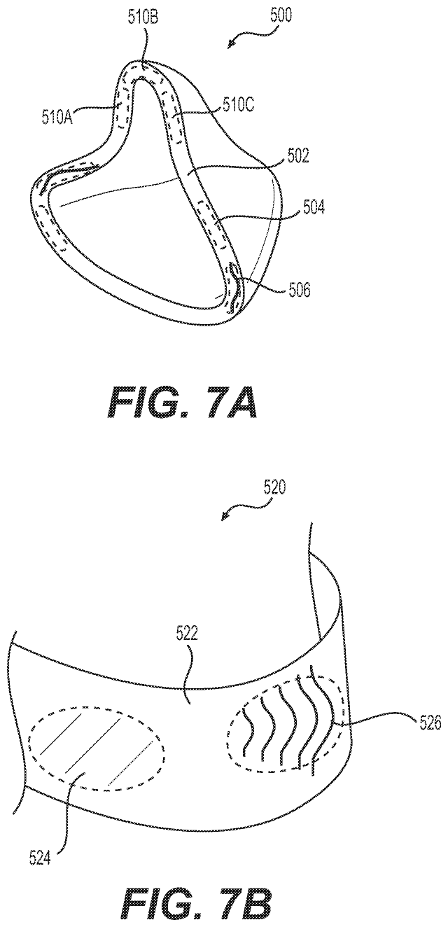

FIG. 7A depicts an example medical device 500 with controllable pressure management elements, in accordance with the present disclosure. In this example, the medical device 500 is a breathing mask that is representative of all devices where the application element is in long-term contact with the skin of a patient. In an aspect, a medical device having an application element in long-term contact with the skin of a patient is a collar or a cast. In one aspect, a medical device having an application element in long-term contact with the skin of a patient is a cervical collar or a cervical cast. In this example, the pressure management elements are inflatable pockets such as pocket 504, which is shown in an inactive, e.g., deflated, state. Pocket 506, by way of comparison, is shown in an active, e.g., inflated, state. When pockets 504, 506 are configured as shown in FIG. 7A, pressure is higher in the region of pocket 506 and lower in the region of pocket 504. In an aspect, the pressure in the region of pocket 504 is low enough to allow blood flow through the tissue of this region.

In an aspect, the pressure management elements are provided in sets such as pockets 510A, 501B, and 510C. These pockets may be manipulated in a coordinated fashion to shift the levels of contact pressure between the device 500 and the skin of the patient in the regions of the pockets 510A, 510B, 510C. For example, the pocket 510B is inflated while pockets 510A, 510C are deflated, creating a relatively high contact pressure area around pocket 510B and a relatively low, e.g. lower than the nominal pressure that would be present in the absence of a pressure management element, contact pressure in the regions of pockets 510A, 510C. This relatively low contact pressure allows adequate blood flow to the tissue in that region so as to avoid tissue damage. At a different time, one or both of pockets 510A, 510C are inflated while pocket 510B is deflated, thus reducing the contact pressure in the region of pocket 510B.

In an aspect, the pockets are flexible membranes that comprise a portion of the walls of a sealed compartment that is within or on the surface of device 500. In an aspect, at least one of the walls of the pockets is stretchable. In one aspect, when the pockets are situated within the surface of device 500, the wall of device 500 that is in contact with the skin of a patient is also stretchable.

The words “force” and “pressure” are considered to be interchangeable within the context of this disclosure. A higher pressure within a pocket will apply a greater pressure over the area of the pocket, which produces a higher total force (pressure×area=force). A greater amount of fluid in the pocket does not intrinsically apply a higher pressure or force; the raised height of the pocket will cause the patient's skin to come in contact with the inflated pocket first and thereby the inflated pocket will provide a greater portion of the total force applied by the device 500 to the patient's skin and such is equivalent to providing a greater pressure and/or force.

Pockets may be fully inflated, fully deflated, or partially inflated to an intermediate pressure. In an aspect, the pockets may be inflated with a gas or a liquid or other fluid. The word “inflation” is interpreted as an indication of pressure or, equivalently, of the amount of fluid within the pocket, such that the phrase “higher inflation” includes the situation of a greater amount of fluid in the compartment.

In an aspect, the pockets are connected to a source of pressurized fluid through elements such as tubing, valves, pressure regulators (not shown in FIG. 7A) that are coupled to and controlled by a controller (not shown in FIG. 7A). In an aspect, the source of pressurized fluid may be the same source of fluid being provided to the patient through the medical device 500, for example pressurized oxygen-enriched air. In an aspect, the controller of the pressure management element is a part of the electronics package 240 of FIG. 4B.

In an aspect, the pressure management element is a mechanical element whose height can be adjusted. In an aspect, the adjustment is provided with an electrical actuator. In an aspect, the actuator comprises a piezoelectric element that causes a change in the height of the element. In an aspect, the pressure management element is a fixed height element that moves parallel to the skin of the patient such that the contact pressure is increased in the region of contact between the element and the skin and reduced in other regions.

FIG. 7B illustrates another medical device that is a strap 520 similar to the strap 414 shown in FIGS. 6A and 6B. In this example, the strap 520 comprises a band 522 with pockets such as pockets 524, 526 spaced along the band 522. In this example, pocket 524 is inactive and pocket 526 is active, causing the contact pressure under pocket 526 to be higher than the contact pressure under pocket 524. In an aspect, the band 522 is overlaid with an array of electrodes 418 (not visible in FIG. 7B) such that strap 520 can both measure SEM and manage the pressure applied by the strap 520 to the patient's skin.

In an aspect, the change in inflation of the pockets is driven by an SEM reading taken, for example, by the electrodes 418 of FIG. 6B. In one aspect, the change in inflation of the pockets is driven by a delta value that is, in an aspect, the difference between the highest SEM value and the lowest SEM value in a set of measurements. In an aspect, a set of measurements includes measurements taken at a single location. In one aspect, a set of measurements includes measurements taken at multiple locations. In one aspect, a set of measurements is taken at approximately the same time, such as within 10 minutes, within 5 minutes, within 1 minute, within 30 seconds, within 10 seconds, within 5 seconds, or within 1 second. In an aspect, a delta value is calculated by the difference between the most recent SEM value and the cumulative average SEM value over a period of time. In one aspect, a cumulative average SEM value is derived from a set of SEM measurements taken since the first use of the medical device. In an aspect, a cumulative average SEM value is derived from SEM measurements taken within approximately a year, such as within 9 months, within 6 months, within 5 months, within 4 months, within 3 months, within 2 months, within 1 month, within four weeks, within three weeks, within two weeks, within one week, within 6 days, within 5 days, within 4 days, within 3 days, within 2 days, within 1 day, within 16 hours, within 12 hours, within 8 hours, within 4 hours, within 3 hours, within 2 hours, within 1 hour, within 45 minutes, within 30 minutes, within 15 minutes, within 10 minutes, or within 5 minutes.

In an aspect, the change in inflation of the pockets is driven by how a calculated delta value is compared to a threshold. When the delta value exceeds the threshold, inflation pattern of the pockets changes to shift the pressure applied to the patients. There may be multiple thresholds used to determine the inflation pattern of the pockets.

In an aspect, the change in inflation is caused by a timer that regularly shifts the pressure applied to the patient by changing the pattern of active pressure management elements, for example by inflating and deflating different pockets.

In an aspect, a series of predetermined configurations of the pressure management elements are defined and the timer configured to execute a programmed series of changes between these configurations at predefined times. In an aspect, the changes between predetermined configurations are based on SEM readings taken of the patient.

In an aspect, there is a configuration of which pockets are inflated and this default is maintained until a SEM reading indicates a problem, whereupon certain pockets are deflated or reduced in inflation height.

From the foregoing, it will be appreciated that the present invention can be embodied in various ways, which include but are not limited to the following:

Embodiment 1. An apparatus for detecting tissue damage proximate to a point of contact between a medical device and a patient's skin, comprising: a first electrode and a second electrode configured to measure a level of sub-epidermal moisture (SEM) in tissue proximate to the point of contact, an electronics package individually connected to the first and second electrodes and configured to measure a capacitance between the first and second electrodes.

Embodiment 2. The apparatus of embodiment 1, where the first and second electrodes are configured to be attached to the medical device.

Embodiment 3. The apparatus of embodiment 1 or 2, where the first and second electrodes are shaped such that the entire surface of each electrode can contact the patient's skin while the medical device is in use.

Embodiment 4. The apparatus of any one of embodiments 1 to 3, further comprising a body coupled to the first and second electrodes, the body configured to be interposed between the medical device and the patient's skin when the medical device is in use.

Embodiment 5. The apparatus of embodiment 4, where the body is further configured to be attached to the medical device.

Embodiment 6. The apparatus of any one of embodiments 1 to 5, further comprising a communication element configured to provide real-time transfer of SEM measurements to a computing unit.

Embodiment 7. The apparatus of any one of embodiments 1 to 6, where the apparatus is a clip configured to attach to a tube of the medical device.

Embodiment 8. The apparatus of embodiment 7, where the tube is selected from the group consisting of a nasogastric tube, a feeding tube, an endotracheal tube, a tracheostomy tube, a tracheostomy collar, a nasal cannula, an IV/PICC line, a catheter, and a fecal management tube.

Embodiment 9. The apparatus of any one of embodiments 1 to 6, where the apparatus is a strap configured to attach to the medical device.

Embodiment 10. The apparatus of embodiment 9, where the medical device is a mask.

Embodiment 11. The apparatus of any one of embodiments 1 to 6, where the medical device is a collar or a cast.

Embodiment 12. The apparatus of any one of embodiments 1 to 11, where the apparatus further comprises one or more pressure management elements.

Embodiment 13. The apparatus of embodiment 12, where each of the one or more pressure management elements is an inflatable pocket.

Embodiment 14. A method for detecting tissue damage proximate to a point of contact between a medical device and a patient's skin, comprising the steps of: measuring a plurality of sub-epidermal moisture (SEM) values of tissue proximate to the point of contact at incremental times, comparing the plurality of SEM values, and determining if there is a significant increase in the SEM that indicates that there is tissue damage.

Embodiment 15. The method of embodiment 14, where there is a significant increase when the largest SEM value of the plurality of SEM values is greater than the smallest SEM value of the plurality of SEM values by an amount that exceeds a threshold.

Embodiment 16. The method of embodiment 14, where there is a significant increase when the largest SEM value of the plurality of SEM values is greater than a threshold.

Embodiment 17. The method of any one of embodiments 14 to 16, where a first measurement of the SEM value is made at the time of the first use of the medical device.

Embodiment 18. The method of any one of embodiments 14 to 17, where the medical device comprises a tube selected from the group consisting of a nasogastric tube, a feeding tube, an endotracheal tube, a tracheostomy tube, a tracheostomy collar, a nasal cannula, an IV/PICC line, a catheter, and a fecal management tube.

Embodiment 19. The method of any one of embodiments 14 to 17, where the medical device is a mask.

Embodiment 20. The method of any one of embodiments 14 to 17, where the medical device is a collar or a cast.

We claim:

1. An apparatus for detecting tissue damage proximate to a point of contact between a medical device and a patient's skin, comprising:
a first electrode and a second electrode configured to measure a level of sub-epidermal moisture (SEM) in tissue proximate to the point of contact,
an electronics package individually connected to the first and second electrodes and configured to measure a capacitance between the first and second electrodes.

2. The apparatus of claim 1, wherein the first and second electrodes are configured to be attached to the medical device.

3. The apparatus of claim 1, wherein the first and second electrodes are shaped such that the entire surface of each electrode can contact the patient's skin while the medical device is in use.

4. The apparatus of claim 1, further comprising a body coupled to the first and second electrodes, the body configured to be interposed between the medical device and the patient's skin when the medical device is in use.

5. The apparatus of claim 4, wherein the body is further configured to be attached to the medical device.

6. The apparatus of claim 1, further comprising a communication element configured to provide real-time transfer of SEM measurements to a computing unit.

7. The apparatus of claim 1, wherein the apparatus is a clip configured to attach to a tube of the medical device.

8. The apparatus of claim 7, wherein the tube is selected from the group consisting of a nasogastric tube, a feeding tube, an endotracheal tube, a tracheostomy tube, a tracheostomy collar, a nasal cannula, an IV/PICC line, a catheter, and a fecal management tube.

9. The apparatus of claim 1, wherein the apparatus is a strap configured to attach to the medical device.

10. The apparatus of claim 9, wherein the medical device is a mask.

11. The apparatus of claim 1, wherein the medical device is a collar or a cast.

12. The apparatus of claim 1, wherein the apparatus further comprises one or more pressure management elements.

13. The apparatus of claim 12, wherein each of the one or more pressure management elements is an inflatable pocket.

14. A method for detecting tissue damage proximate to a point of contact between a medical device and a patient's skin, comprising the steps of:
measuring a plurality of sub-epidermal moisture (SEM) values of tissue proximate to the point of contact at incremental times,
comparing the plurality of SEM values, and
determining if there is a significant increase in the SEM that indicates that there is tissue damage.

15. The method of claim 14, wherein there is a significant increase when the largest SEM value of the plurality of SEM values is greater than the smallest SEM value of the plurality of SEM values by an amount that exceeds a threshold.

16. The method of claim 14, wherein there is a significant increase when the largest SEM value of the plurality of SEM values is greater than a threshold.

17. The method of claim 14, wherein a first measurement of the SEM value is made at the time of the first use of the medical device.

18. The method of claim 14, wherein the medical device comprises a tube selected from the group consisting of a nasogastric tube, a feeding tube, an endotracheal tube, a tracheostomy tube, a tracheostomy collar, a nasal cannula, an IV/PICC line, a catheter, and a fecal management tube.

19. The method of claim 14, wherein the medical device is a mask.

20. The method of claim 14, wherein the medical device is a collar or a cast.
